(19) United States
(12) Patent Application Publication
(10) Pub. No.: US 2025/0235648 A1
(43) Pub. Date: Jul. 24, 2025

(54) PATIENT INTERFACE CUSHION

(71) Applicant: DCSTAR INC, New York, NY (US)

(72) Inventor: David LUO, New York, NY (US)

(21) Appl. No.: 18/416,124

(22) Filed: Jan. 18, 2024

(51) Int. Cl. A61M 16/06

(52) U.S. Cl. CPC A61M 16/0622; A61M 16/0683

(57) ABSTRACT

A patient interface cushion includes a rigid section and an elastic section. The elastic section is divided into a main body of the elastic section, which at least partially contacts and forms a seal with the patient's face during use, and a connecting portion to connect to the rigid section. The main body of the elastic section has multiple areas to enable the patient interface cushion to have different degrees of variation in different areas and to adjust the pressure exerted by the patient interface cushion on different facial areas. It specifically includes an adjustment area to accommodate different face sizes, nose heights, and widths; a support area to support the patient interface cushion; and a stabilization area to prevent the patient interface cushion from turning out. The adjustment area can be configured to include a nasal side adjustment part.

### TECHNICAL FIELD

This disclosure pertains to a patient interface cushion for Continuous Positive Airway Pressure (CPAP) therapy, configured to deliver breathable pressurized gas to a patient for the treatment of sleep breathing disorders. The patient interface cushion includes a rigid section and an elastic section. It is applicable for one or more of the treatment and improvement of respiratory diseases.

### BACKGROUND

Sleep Apnea Syndrome (SAS), also known as Sleep Apnea Hypopnea Syndrome, is a common syndrome in modern society. It can occur in anyone regardless of age, gender, or race, and is globally prevalent. According to data from the World Health Organization, as of 2022, over a billion people worldwide suffer from SAS, with the number continually rising, possibly related to population aging and increasingly unhealthy lifestyles. Although SAS can occur in anyone, there are differences in incidence rates between males and females, and across different age groups. Typically, males are more prone to the condition, especially in the middle-aged and older population. SAS is currently categorized into Obstructive Sleep Apnea (OSA), Central Sleep Apnea (CSA) caused by central nervous system anomalies, and Mixed Sleep Apnea Syndrome (MSAS) combining symptoms of both types, with OSA being the most common one. Common symptoms of OSA include loud and irregular snoring during sleep, intermittent nocturnal awakenings, dry mouth, and sore throat due to airway obstruction, excessive daytime sleepiness and severe sleep deprivation caused by interrupted sleep. The most prevalent symptom of OSA is apnea during sleep, lasting from several seconds to minutes. OSA can lead to many complications that disrupt lives, such as hypertension, arrhythmia, myocardial infarction, and heart failure, as well as diabetes and pulmonary diseases. OSA impacts patients' lives through its nocturnal symptoms and complications, affecting health, economy, productivity, traffic safety, sleep, mental health, etc. Patients with OSA should seek treatment or improvement to reduce its impact on life and other aspects.

The treatment for patients with moderate and severe OSA typically involves the use of a ventilator, an essential component of which is the patient interface cushion. The patient interface cushion is configured to fit the patient's face. During use, one end of the patient interface cushion connects to an air supply tube or an elbow, and the other end contacts and seals with the patient's face, to deliver a continuous positive pressure airflow which maintains the patient's airway open, into the nasal airway or oral airway. In response to high demand for patient interface cushions, the market now offers various styles and models of patient interface cushions. However, existing patient interface cushions still leave room for improvement in various areas, such as the feeling of pressure they exert on the patient's face, the tendency to leave indentation and red marks especially in tightly fitting areas after prolonged wear, inability to adapt to all patients' facial contours and sizes, and inability to accommodate all patients' nose heights and widths, and cleaning difficulties. Yet, no existing patient interface cushion on the market solves all these issues to provide a better wearing experience for the patients.

### SUMMARY

This disclosure addresses the problems of existing patient interface cushions and, through continuous research and innovation, introduces a patient interface cushion that resolves the issues above. It offers an improved patient interface cushion, with advantages over existing products in terms of wearing comfort, seal, adaptability, compatibility, and ease of cleaning. This product is more competitive in the current market and can help patients better accept CPAP treatment, ensuring the continuity of treatment and patient compliance.

The objective of this disclosure is to provide a new type of patient interface cushion that offers a more comfortable wearing experience for patients, ensures higher effectiveness for the treatment of sleep apnea, and overcomes the limitations of existing similar products. In this way, the headgear provided by this disclosure is more effective, applicable in a broader range of scenarios, along with more accessible methods, delivering pressurized gas from the ventilator to the patient's airway to treat sleep breathing disorders.

In one embodiment, a patient interface cushion is provided, configured to deliver breathable pressurized gas to a patient to treat sleep breathing disorders, the patient interface cushion including at least some of the following elements or features.

A rigid section has an air intake opening to receive breathable pressurized gas and is configured to connect to an elastic section at one end.

The elastic section, integrally formed with the rigid section, includes a main body of the elastic section that at least partially contacts and forms a seal with the patient's face during use, and a connecting portion to connect to the rigid section.

The main body of the elastic section has at least three different wall thicknesses, the main body of the elastic section at least including: an adjustment area, with an opening near the face of the patient and an edge portion near the opening, the edge portion near the opening has at least part of the wall thickness not exceeding 0.6 mm; a support area, configured to connect to the connecting portion of the elastic section; and a stabilization area, near and connected to the support area, having a wall thickness thicker than at least part of the wall thickness of the support area.

In one embodiment, the support area has at least two different wall thicknesses, and a part near the connecting portion has a thicker wall thickness for support.

In one embodiment, the adjustment area, during use, surrounds the patient's nasal airway and oral airway.

In one embodiment, the stabilization area is an area with the least proportion in the main body of the elastic section of the patient interface cushion.

In one embodiment, each area of the main body of the elastic section is smoothly connected, without any obvious demarcation lines.

In another embodiment, a patient interface cushion is provided, configured to deliver breathable pressurized gas to a patient to treat sleep breathing disorders, the patient interface cushion including at least some of the following elements or features.

A rigid section has an air intake opening to receive breathable pressurized gas and is configured to connect to an elastic section at one end.

The elastic section with a single-layer wall, integrally formed with the rigid section, includes a main body of the elastic section that at least partially contacts and forms a seal with the patient's face during use, and a connecting portion to connect to the rigid section.

The main body of the elastic section includes: an adjustment area, with an opening near the patient's face and an edge portion near the opening; a support area, configured to connect to the connecting portion of the elastic section, with a wall thickness between 2 to 9 times the wall thickness of the adjustment area; and a stabilization area, near and connected to the support area, with a wall thickness between 3 to 10 times the wall thickness of the adjustment area.

In one embodiment, the wall thickness of the adjustment area is at least 0.3 mm.

In one embodiment, a width of the edge portion of the adjustment area is at most 2 cm.

In one embodiment, each area of the main body of the elastic section has a uniform wall thickness.

In one embodiment, the main body of the elastic section has abrupt changes in wall thicknesses between its areas, with distinct demarcation lines between each area.

In yet another embodiment, a patient interface cushion is provided, configured to deliver breathable pressurized gas to a patient to treat sleep breathing disorders, the patient interface cushion including at least some of the following elements or features.

A rigid section has an air intake opening to receive breathable pressurized gas and is configured to connect to an elastic section at one end.

The elastic section, integrally formed with the rigid section, includes a main body of the elastic section that at least partially contacts and forms a seal with the patient's face during use, and a connecting portion to connect to the rigid section.

The main body of the elastic section has at least three different wall thicknesses and at least two surface treatments. The main body of the elastic section at least includes: an adjustment area, with an opening near the patient's face and an edge portion near the opening, at least part of the edge portion has a surface treatment of polishing; a support area, configured to connect to the connecting portion of the elastic section, with a wall thickness thicker than that of the adjustment area at a top near the adjustment area to prevent the patient's nose from bottoming out during use; and a stabilization area, near and connected to the support area, with a wall thickness thicker than that of the adjustment area.

In one embodiment, the support area has a gradually varying wall thickness at the top near the adjustment area, with a thicker wall thickness near the connecting portion.

In one embodiment, a maximum width of the adjustment area at a top of the patient interface cushion to accommodate a nose does not exceed 5.5 cm.

In one embodiment, part of the adjustment area that contacts the patient's nose during use has an inward concave, configured to better accommodate the patient's nose and form a seal.

In one embodiment, the stabilization area is an area with the least proportion in the main body of the elastic section of the patient interface cushion.

In another embodiment, a patient interface cushion is provided, configured to deliver breathable pressurized gas to a patient to treat sleep breathing disorders, the patient interface cushion including at least some of the following elements or features.

A rigid section has an air intake opening to receive breathable pressurized gas and is configured to connect to an elastic section at one end.

The elastic section, integrally formed with the rigid section, includes a main body of the elastic section that at least partially contacts and forms a seal with the patient's face during use, and a connecting portion to connect to the rigid section.

The main body of the elastic section has at least three different wall thicknesses, the main body of the elastic section including: an adjustment area, with an opening near the patient's face and an edge portion near the opening; a support area, configured to connect to the connecting portion of the elastic section and having a gradually varying wall thickness at a region near the connecting portion; and a stabilization area, near and connected to the support area.

In one embodiment, the adjustment area includes a nasal side adjustment part with a wall thickness between the wall thickness of the edge portion and the support area.

In one embodiment, a width of the edge portion of the adjustment area is at most 2 cm.

In one embodiment, the stabilization area is an area with the least proportion in the main body of the elastic section of the patient interface cushion.

In one embodiment, the main body of the elastic section has abrupt changes in wall thicknesses between its areas, with distinct demarcation lines between each area.

In yet another embodiment, a patient interface cushion is provided, configured to deliver breathable pressurized gas to a patient to treat sleep breathing disorders, the patient interface cushion including at least some of the following elements or features.

A rigid section has an air intake opening to receive breathable pressurized gas and is configured to connect to an elastic section at one end.

The elastic section with a single-layer wall, integrally formed with the rigid section, includes a main body of the elastic section that at least partially contacts and forms a seal with the patient's face during use, and a connecting portion to connect to the rigid section.

The main body of the elastic section includes: an adjustment area, with an opening near the patient's face and an edge portion near the opening, and a nasal side adjustment part near the edge portion with a wall thickness between the wall thickness of the edge portion and the support area, with the adjustment area having at least part of a wall thickness of at least 0.3 mm; a support area, configured to connect to the connecting portion of the elastic section; and a stabilization area, near and connected to the support area.

In one embodiment, the stabilization area has a wall thickness thicker than at least part of the wall thickness of the adjustment area and the support area.

In one embodiment, the support area has at least two different wall thicknesses, and a part near the connecting portion has a gradually varying wall thickness for support.

In one embodiment, the elastic section has at least three different wall thicknesses.

In one embodiment, each area of the main body of the elastic section is smoothly connected, without any obvious demarcation lines.

The benefits of a patient interface cushion provided by this disclosure can at least include:
1) By testing the pressure values and sensitivity of different facial areas, combined with the seal of the patient interface cushion, different areas are defined in the main body of the elastic section, each having specific functions. This provides higher comfort and a better user experience for the patient, thus improving their compliance with the treatment. Through research on platforms like CPAP forums, Reddit, Amazon customer review sections, YouTube, etc., it was found that most existing patient interface cushions on the market cause discomfort or leakage during wear. During wear, discomfort in patients is caused not only by the material of the patient interface cushion but also by its inability to fully conform to the patient's face. As different areas of the face have distinct contour curves, existing patient interface cushions on the market cannot adjust and vary according to these facial contour curves. Consequently, they fail to provide satisfactory comfort and pressure for the patient. This inadequacy in fitting also leads to insufficient sealing of the patient interface cushions. When the same amount of pressure is applied to the elastic section of the patient interface cushion, the elastic section deforms uniformly to fit the face. However, due to the varying contour curves of the face, this uniform deformation of the elastic section cannot achieve a complete fit with the patient's face, resulting in air leakage. And existing patient interface cushions on the market have parts that are easily blown over, so the parts were thickened to prevent turning out and leakage. In summary, based on precise experimental data, this disclosure divides the main body of the elastic section of the patient interface cushion, which has the most contact with the patient's face, into at least three areas: an adjustment area, a stabilization area, and a support area. The adjustment area is configured to increase compatibility with the patient's face and nose, the stabilization area is thickened to prevent the elastic section from turning out, and the support area is to support the patient interface cushion. To ensure the supportive function of the elastic section of the patient interface cushion, the support area has at least two different thicknesses, with the part near the connecting portion being thicker and variable in wall thicknesses, and the part nearer the patient's face being thinner during use.
2) In the adjustment area, a thinner edge portion is set to increase compatibility with the patient's face, making the elastic section of the patient interface cushion fit more closely to the patient's face and enhancing the sealing. Based on the above experiments, this disclosure noted that the main body of the elastic section of the patient interface cushions available on the market greatly impacts the overall sealing and comfort of the cushion, particularly in the edge portion near the opening facing the patient's face. Therefore, the edge portion has been reconfigured in the disclosure to improve the comfort and sealing while making the patient interface cushion more suitable for most patients. a) From some Amazon buyer reviews and comments on other platforms, this disclosure found that although existing patient interface cushions on the market come in S, M, L sizes, due to differences in race and weight, some patients still cannot find a cushion that fits their face size, with the L size causing gas leakage and the M size creating discomfort due to pressure. Hence, the disclosure has made the edge portion near the opening facing the patient's face of the main body of the elastic section of the patient interface cushion thinner to accommodate more face shapes. With the thinner edge portion that can easily fold inward, patients with smaller faces can directly use the inner edge as the sealing edge under the pressurized gas, while patients with larger faces can press down the edge portion of the patient interface cushion and use the edge of the support area near the edge for sealing. This disclosure adapts the patient interface cushion to a range of face sizes instead of a specific value, facilitating the selection for patients who cannot find a suitable size among various brands, thereby expanding the compatibility of the patient interface cushion. b) In addition to increasing compatibility, setting the edge portion to be thinner also enhances the sealing of the patient interface cushion. The design which makes the edge portion thin and of a uniform thickness allows the edge portion to be more easily lifted by airflow during use, facilitating contact with the patient's face to form a seal, making the seal more stable. The thinner edge portion has greater flexibility and can better adapt to different facial contours. Uniform thickness means that the force exerted on the patient's face by the inner edge of the patient interface cushion is more even, preventing excessive pressure points or discomfort, thereby providing more uniform comfort. Also, the uniform thickness can accommodate more facial contour shapes, fitting a wider range of people. c) This disclosure uses a special surface treatment process, such as, polishing of the edge portion. A polished surface is smoother than other surfaces and has greater adhesion to the skin during use, thus achieving a better and more stable fit. Moreover, a polished surface is more uniform, without depressions or protrusions, increasing the sealing between the elastic section of the patient interface cushion and the patient's face and reducing gas leakage. To ensure comfort, the patient interface cushion may also have a relatively rough frosted surface, which has less adhesion to the skin during use, increasing comfort for the patient wearing the patient interface cushion. This disclosure combines two surface treatments to ensure better sealing while maintaining the comfort of the patient interface cushion.
3) The design of the area near the nasal airway is easily adjustable to increase the accommodation of the patient's nose and prevent bottoming out. One of the significant differences in human faces lies in the height and width of the nose. Some people have higher nose bridges, while others have lower ones; some have narrower noses, and others have wider noses. Such differences lead to the necessity of adapting the patient interface cushion to a variety of nose heights and widths. The design of the area at which the main body of the elastic section contacts the patient's alae nasi directly affects the fit and sealing effect of the patient interface cushion with the patient's face and it should be carefully considered. The patient interface cushion in this disclosure has at least two different wall thicknesses in the area where the main body of the elastic section contacts the patient's alae nasi and nasal sides. This design allows greater accommodation for the nose during use, adapting to various nose heights and widths, and further increasing the compatibility of the patient interface cushion with the patient's face, thus providing better treatment efficacy. Specifically, a) when patients use the patient interface cushion of this disclosure, the area of the main body of the elastic section that has the most contact with the patient's nose is the adjustment area with a thinner wall thickness. Its thinner wall can more easily conform to the patient's nose. The adjustment area can easily deform according to the height of the nose due to the pressure from the nose, and it has a thinner wall thickness, which brings greater flexibility and less pressure on the nose, improving comfort during use. b) The support area, near the top of the adjustment area, has a thicker wall thickness than that of the adjustment area. It is configured to prevent the elastic section from bottoming out due to excessive pressure from the patient's nose, providing greater support for the nose and reducing discomfort caused by the patient interface cushion. c) The adjustment area near the patient's nose also includes a nasal side adjustment part. To address the issue of patient interface cushions not fitting patients with different nose widths, the nasal side adjustment part enhances the compatibility of the patient interface cushion with the patient's nose. Under the pressurized gas, patients with narrower noses can use the boundary of the thinner area as the outer contour of the elastic section of the patient interface cushion to accommodate the nose and change the shape. In contrast, patients with wider noses can press down the nasal side adjustment part, using part of or the entire boundary of the nasal side adjustment part as the outer contour of the elastic section of the patient interface cushion to accommodate the nose and change the shape. This design adapts to patients with different nose widths, ensures a better fit of the patient interface cushion with the patient's face and meets the needs of various patients.
4) Compared to patient interface cushions with straps and dual-layer designs, this disclosure maintains the original functionality while simplifying the mold, reducing the defect rate, and lowering production costs. It also offers benefits in terms of environmental protection and ease of cleaning. Most existing patient interface cushions on the market have a double-layer structure or straps to prevent the elastic section from turning out. While these designs solve the issue of the elastic section turning out, they introduce several problems. For example, double-layered patient interface cushions are heavier than single-layered ones, which can cause discomfort for patients, especially during long periods of wear. Both double-layered patient interface cushions and ones with straps are more expensive in terms of material costs, require more complex molds leading to higher mold costs, and involve more complicated manufacturing processes. Additionally, due to the complexity of their processes, the likelihood of producing defective products increases during manufacturing, which further elevates manufacturing costs and may result in increased labor costs due to quality control and sorting. The patient interface cushion provided by this disclosure uses the stabilization area of the main body of the elastic section to replace the structure of double-layered patient interface cushions or ones with straps, achieving the same functionality but with simpler, more cost-effective processes and a higher yield of quality products. This design demonstrates better features compared to existing patient interface cushions in various aspects and meets the requirements of green design, having beneficial effects on climate change and environmental protection. In terms of cleaning, double-layered patient interface cushions or ones with straps, being made of multiple layers of material, may have corners that easily accumulate dirt and are hard to clean. Over time, this can lead to the growth of bacteria and other microbes, adversely affecting patient health. The cleaning process may require more time and possibly more frequent cleaning sessions to ensure thoroughness. Moreover, during the use of ventilators for the treatment of OSA, the patient interface cushions tend to accumulate moisture. The multi-layer material impedes evaporation of the moisture and requires longer drying times, which is not conducive to multiple or prolonged usage by patients and is another factor detrimental to patients' health.

### BRIEF DESCRIPTION OF THE DRAWINGS

FIG. 1 is a perspective schematic diagram of a patient interface cushion in accordance with an embodiment;

FIG. 2 is a front-view diagram showing various regions of a patient interface cushion in accordance with an embodiment;

FIG. 3 is a side-view diagram showing various regions of a patient interface cushion in accordance with an embodiment;

FIG. 4 is a schematic rear-view diagram having sectional lines of a patient interface cushion in accordance with an embodiment;

FIG. 5 is a cross-sectional structure schematic diagram along line 5-5 of FIG. 4;

FIG. 6 is a cross-sectional structure schematic diagram along line 6-6 of FIG. 4;

FIG. 7 is a cross-sectional structure schematic diagram along line 7-7 of FIG. 4;

FIG. 8 is a schematic diagram of developing a patient interface cushion in accordance with an embodiment;

FIG. 9 is a diagram showing the stress distribution on a patient interface cushion in accordance with an embodiment;

FIG. 10 is a diagram showing changes in the edge portion of a patient interface cushion when used by patients with different facial sizes in accordance with an embodiment;

FIG. 11 is a schematic diagram showing a patient interface cushion having a polishing treatment on an edge portion in accordance with an embodiment;

FIG. 12 is a diagram showing changes in the adjustment area of a patient interface cushion when used by patients with different nose heights and widths in accordance with an embodiment;

FIG. 13 is a schematic diagram illustrating a patient interface cushion preventing the nose from bottoming out in accordance with an embodiment;

FIGS. 14A and 14B are schematic diagrams illustrating a different form of the regional division of a patient interface cushion in another implementation in accordance with an embodiment;

FIG. 15 is a schematic diagram illustrating the elastic section of a patient interface cushion separated from the rigid section and connected using physical clips in another implementation in accordance with an embodiment;

FIGS. 16A and 16B are schematic diagrams of a patient interface cushion with an exhaust port or a noise reduction component in another implementation in accordance with an embodiment;

FIGS. 17A and 17B are schematic diagrams of the patient interface cushion with a comfort layer in another implementation in accordance with an embodiment;

FIG. 18 is a schematic diagram illustrating the use of the elastic section in another form of a patient interface cushion in accordance with an embodiment;

FIG. 19 is a schematic diagram showing the use of the elastic section in another form of a patient interface cushion in another implementation in accordance with an embodiment;

FIG. 20 is a schematic diagram of different forms of a stabilization area on a patient interface cushion in accordance with an embodiment;

FIG. 21 is a schematic diagram of different forms of the adjustment area of a patient interface cushion in another implementation in accordance with an embodiment;

FIGS. 22A, 22B and 22C are schematic diagrams illustrating different forms of the support area of a patient interface cushion in another implementation in accordance with an embodiment.

### DETAILED DESCRIPTION

To facilitate the understanding of the disclosure, a more comprehensive description will be provided with reference to the relevant drawings. The drawings illustrate typical embodiments of the disclosure. However, the disclosure can be implemented in many different forms and is not limited to the embodiments described herein. On the contrary, the embodiments are provided to make the disclosure more thorough and comprehensive.

Unless otherwise defined, all technical and scientific terms used herein have the same meaning as commonly understood by one of ordinary skill in the art to which this disclosure belongs. The terms used in the specification of the disclosure herein are for the purpose of describing particular embodiments only rather than limiting the disclosure.

To address the issues of insufficient seal and comfort, production difficulty, and high cost of existing patient interface cushions used for delivering gas to patients' airways, this disclosure provides a patient interface cushion with a simpler structure and improved wearing comfort. The disclosure not only optimizes existing designs but also offers patient interface cushions with several different structures for patients to choose according to their needs and preferences. This represents a better technical disclosure for users, producers, and the market. Besides, the design is also sustainable and environmentally friendly since it reduces the waste of materials.

Detailed embodiments are presented below to elucidate several structures of a patient interface cushion.

### Embodiment 1

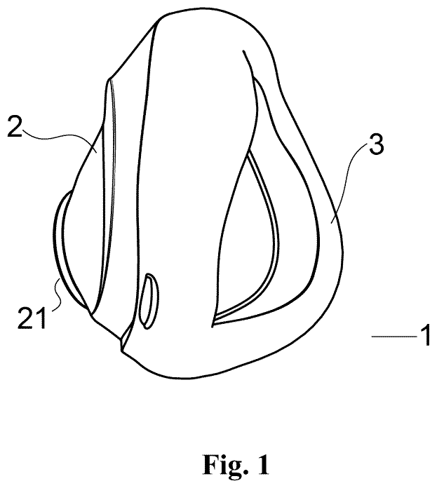

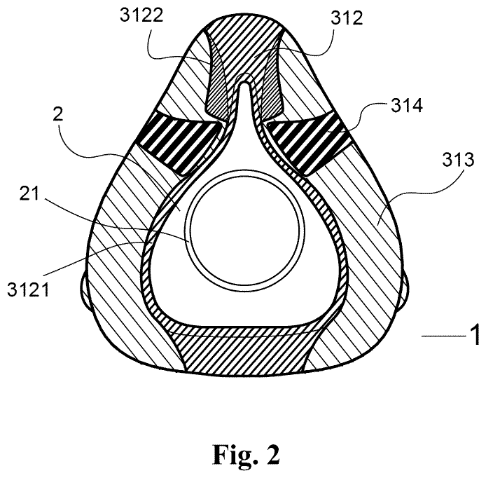

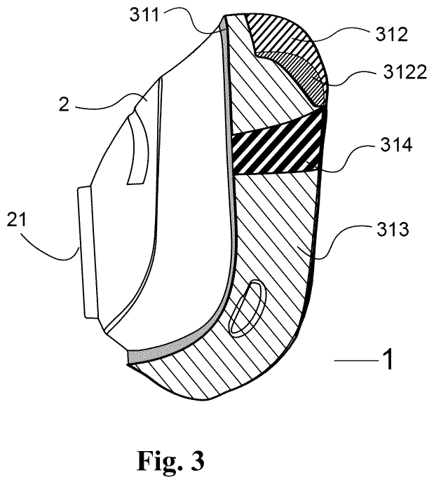

This embodiment provides a patient interface cushion 1 for use in the CPAP field, configured to deliver breathable pressurized gas to patients for the treatment of sleep breathing disorders. This embodiment includes a three-dimensional schematic, diagrams for various regions, cross-sectional structure schematic diagrams, an experimental schematic diagram and a diagram showing the stress distribution of the patient interface cushion 1, as shown in FIGS. 1-13. The patient interface cushion 1 in this embodiment comprises a rigid section 2 and an elastic section 3. The elastic section 3 includes a main body 31 of the elastic section and a connecting portion 311 that connects to the rigid section 2. The main body 31 of the elastic section at least includes an adjustment area 312, a support area 313, and a stabilization area 314, each with corresponding functions configured to enhance the patients' comfort, the sealing of the patient interface cushion 1, and its compatibility with the patient's face.

The patient interface cushion 1 includes a rigid section 2 with an air intake opening 21 to receive breathable pressurized gas and is configured to connect to the elastic section 3 at one end. The air intake opening 21 may be an opening formed by a wall extending towards or away from the patient's face and is configured to connect to a tube, a frame, or an elbow that delivers breathable pressurized gas through means such as snap-fit, a clip, or magnetic attraction. The rigid section 2 provides the necessary structural stability to the patient interface cushion 1, ensuring the shape of the patient interface cushion 1 remains unchanged during wear, thereby maintaining its primary function. The material of the patient interface cushion 1 can be high-density polyethylene, polypropylene, polycarbonate, polystyrene, etc.

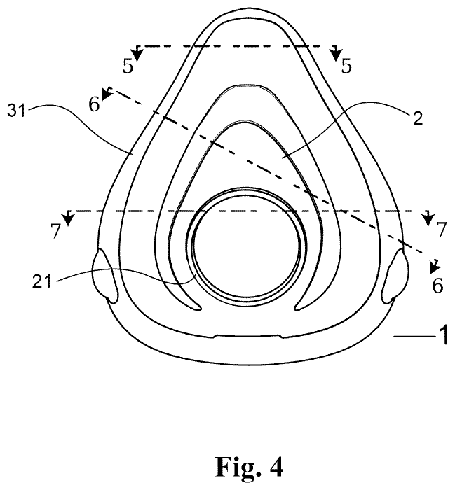

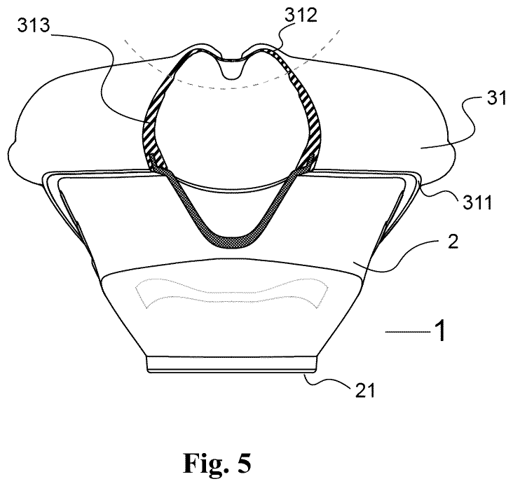

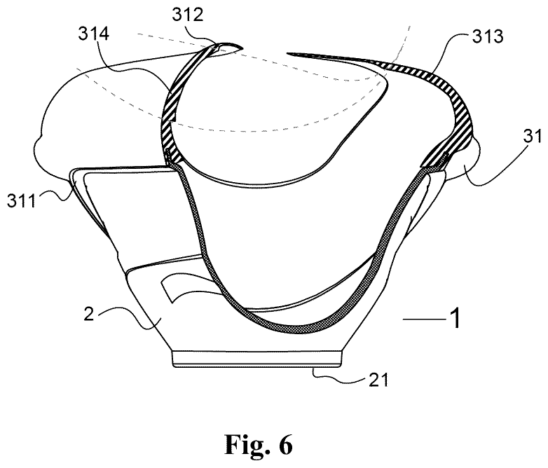

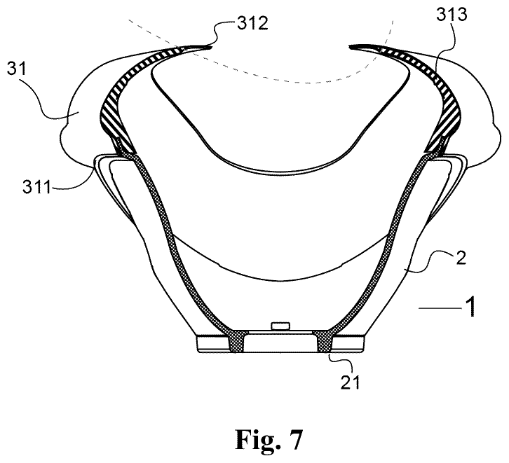

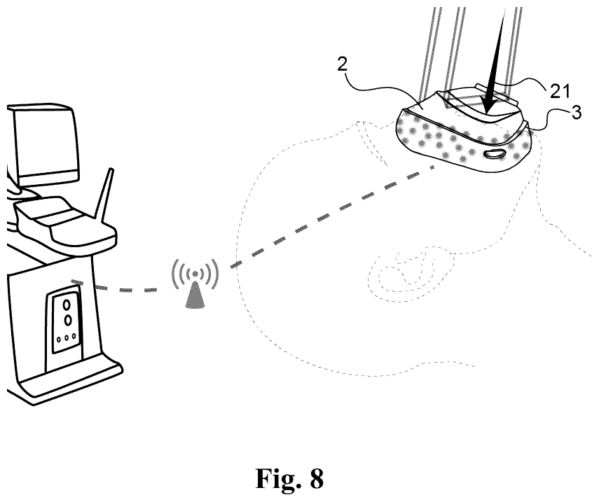

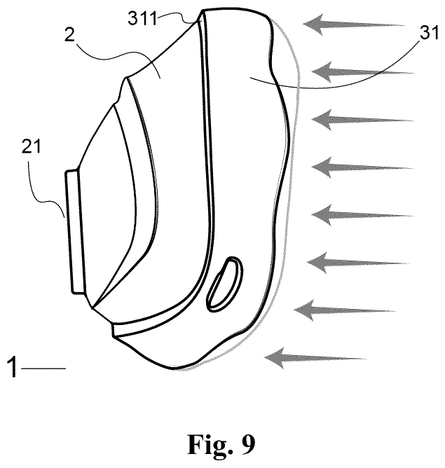

The elastic section 3, integrally formed with the rigid section 2, includes a main body of the elastic section 31, which at least partially contacts and forms a seal with the patient's face during use, and a connecting portion 311 that connects to the rigid section 2. The elastic section 3 is made of a material softer than that of the rigid section 2, such as silicone, rubber, or elastic plastic. During use, the elastic section 3 is configured to connect to the rigid section 2 using the connecting portion 311 through flexible clips, adhesives, magnetic attraction, or co-molding. During use, at least part of the main body 31 of the elastic section contacts the patient's face to distribute the pressure exerted by the patient interface cushion 1 on the face. The design of the main body 31 of the elastic section enhances the performance of the patient interface cushion 1, improves the comfort, compatibility, and sealing of the patient interface cushion 1, ensuring a better experience during treatment, reducing discomfort, and thus improving compliance and efficacy of the treatment. The main body 31 of the elastic section is configured to be a single-layer wall, which achieves the same effect as a double-layer wall, facilitates cleaning of the patient interface cushion 1, reduces the weight of the patient interface cushion 1, avoids discomfort during prolonged wear, and saves material and mold costs. This design also simplifies the process and reduces the defect rate, with the advantage of being easy to dry and clean without harboring bacteria. Experiments and tests with different types and sizes of patient interface cushions 1 were conducted using the patient interface cushion on patients' faces. The steps are as follows: a) Prepare the equipment and material as needed, including a patient head model with standard dimensions, different types and sizes of patient interface cushions, pressure sensors (which can be silicon-based or flexible pressure sensors), a pressure device, and a computer or a data collection and analysis system; b) Place the pressure sensors as densely as possible on various areas of the face of the patient head model, as shown in FIG. 8 (or wear the pressure sensors on the face of the patient head model), fix different types or sizes of patient interface cushions on the face of the patient head model, apply the same pressure using a standard pressure device (pressure fixtures or other equipment), and record the data from the pressure sensors in each facial area, then perform repeated tests and data recording; c) Analyze and calculate the average pressure values in each facial area of the patient head model to adjust the size and wall thickness of the patient interface cushion in different facial areas, thereby addressing issues of patient interface cushions not sealing due to uneven fitting in various facial areas and discomfort due to uneven pressure distribution. As shown in FIG. 9, with the same pressure applied to the patient interface cushion in the same direction and with the same wall thickness, each facial area exerts different pressures on the patient interface cushion, with different degrees of deformation. From data analysis and conclusions, the wall thickness of the area in the main body of the elastic section of the patient interface cushion corresponding to the area of the patient head model with the highest average pressure is reduced, allowing the facial area to comfortably conform to the corresponding position of the patient interface cushion, while the wall thickness of the areas corresponding to areas with lower average pressure on the patient head model is configured to be of appropriate thickness, providing a better user experience and reinforcing the patient interface cushion to prevent leakage. The experiments concluded that the elastic section 3 needs at least three different wall thicknesses. Accordingly, the main body 31 of the elastic section of the patient interface cushion 1 is divided into at least three areas: an adjustment area 312, a support area 313, and a stabilization area 314. As shown in FIG. 4, there is a schematic rear-view diagram having sectional lines of a patient interface cushion 1 showing the different wall thicknesses of these three areas. Specifically, FIG. 5 shows the 5-5 sectional view from FIG. 4, which displays the wall thicknesses of the adjustment area 312 and the stabilization area 314 of the patient interface cushion 1. FIG. 6 corresponds to the 6-6 sectional view in FIG. 4, illustrating the wall thicknesses of the adjustment area 312, the support area 313, and the stabilization area 314 of the patient interface cushion 1. FIG. 7 is the 7-7 sectional view from FIG. 4, showcasing the wall thicknesses of the adjustment area 312 and the support area 313. Each area of the main body 31 of the elastic section can have a uniform wall thickness or a non-uniform wall thickness such as a gradually varying wall thickness, with each area of the elastic section 3 being smoothly connected, employing a uniform transition to achieve variations in wall thicknesses. There are no obvious demarcation lines between each area (no steps, and the changes in wall thicknesses are not visible to the naked eye). Alternatively, the areas of the elastic section 3 can have abrupt changes (forming height differences visible to the naked eye, e.g., steps) in wall thicknesses, resulting in clear demarcation lines between each area.

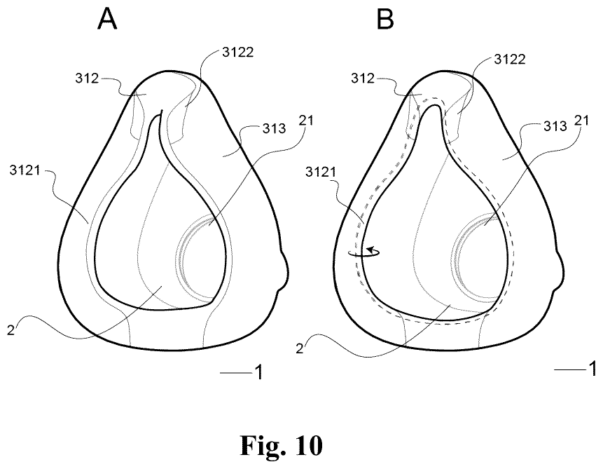

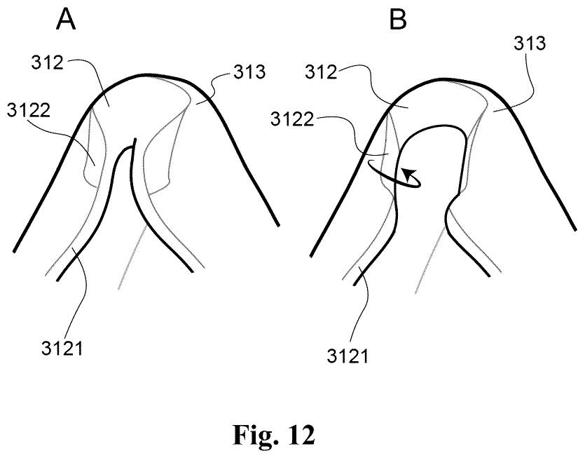

The main body 31 of the elastic section has at least three different wall thicknesses, at least including an adjustment area 312 with an opening near the patient's face and with an edge portion 3121 near the opening. The adjustment area 312 surrounds the patient's nasal and oral airways during use and is adjustable to accommodate different face sizes and nose heights and widths. The maximum width of the adjustment area at the top of the patient interface cushion 1 to accommodate the nose does not exceed 5.5 cm (including the edge portion 3121 at the top and the nasal side adjustment part 3122). The part that can be adjusted to the face sizes of different patients is the edge portion 3121. The edge portion 3121, configured to be thinner, can easily fold inward to fit different face sizes. Specifically, for patients with smaller faces, the edge portion 3121 can be blown up by pressurized gas to use the inner edge as the sealing edge under the pressurized gas (as shown in FIG. 10A); for patients with larger faces, the edge portion 3121 can be pressed down, using the edge of the support area 313 near the edge port 3121 for sealing (as shown in FIG. 10B, the solid line represents the boundary of the seal, and the broken line shows the inwardly folded edge). The edge portion 3121 near the opening has at least part of the wall thickness of no more than 0.6 mm, and the adjustment area 312 has at least part of the wall thickness of at least 0.3 mm. The design of the edge portion 3121 as a thinner area is also intended to achieve better sealing. In this way, the edge portion 3121 can be easily blown up by airflow, achieving better sealing and forming a more stable seal with the patient's face. For sealing and comfort, preferably, the width of the edge portion 3121 of the adjustment area 312 is set to a maximum of 2 cm. Moreover, the edge portion 3121 has a uniform thickness that enhances the comfort of the patient interface cushion 1, ensuring even contact with the patient's face without excessive pressure points. The main body 31 of the elastic section may also have at least two surface treatments. The edge portion 3121 has a special surface treatment, specifically polishing at least part of the edge portion 3121, making the polishing surface smoother and stickier to the skin for a more stable fit. To ensure comfort, the patient interface cushion 1 (e.g., in the area that is relatively flat on the cheek) also has a relatively rough frosted surface (non-smooth surface, causing light to scatter diffusely when hitting the surface). Combining these two surface treatments ensures good sealing and maintains the comfort of the patient interface cushion 1. The part that can be adjusted according to the heights and widths of the patient's nose is an adjustment area 312 on the top of the patient interface cushion 1, including a nasal side adjustment part 3122. The adjustment area 312 has at least two thicknesses to better accommodate the noses. Specifically, the adjustment area 312 has a nasal side adjustment part 3122 near the edge portion 3121, with a wall thickness between the edge portion 3121 and the support area 313. Within the adjustment area 312 on the top of the patient interface cushion 1, the nasal side adjustment part 3122 is of a thicker wall thickness, while the other parts (the adjustment area 312 excluding the area of the nasal side adjustment part 3122) are of a thinner wall thickness. During use, for patients with narrower noses, the edge portion 3121 and the nasal side adjustment part 3122 are blown up by the pressurized gas to seal the nose (as shown in FIG. 12A), using the thinner wall boundary as the sealing edge of the elastic section 3 of the patient interface cushion 1. For patients with wider noses, the nasal side adjustment part 3122 can be pressed down, using part of the nasal side adjustment part 3122 or the boundary of the nasal side adjustment part 3122 near the support area 313 as the sealing edge of the elastic section 3 of the patient interface cushion 1 to seal the nose (as shown in FIG. 12B). This accommodates patients with different nose widths and ensures a better fit of the patient interface cushion 1 to the patient's face, meeting the needs of different patients. The nasal side adjustment part 3122 can have either a uniform wall thickness or a gradually varying wall thickness. Additionally, the adjustment area 312, when in use, has an inward depression where it contacts the patient's nose (as shown in FIG. 12), configured to better accommodate and seal the nose.

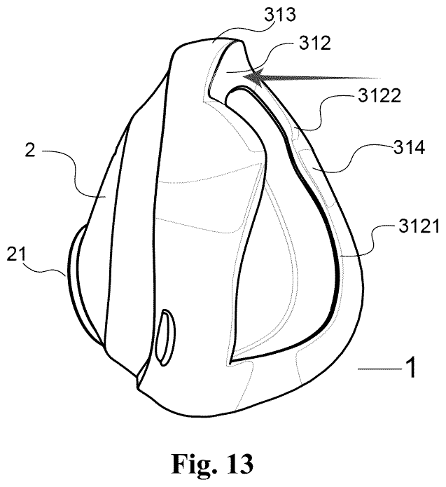

The support area 313 is configured to connect to the connecting portion 311 of the elastic section 3. The support area 313 is the largest area among all sections and, at least partially, comes into contact with the patient's face during use, providing substantial support to the face. Experimental tests have demonstrated that the most suitable thickness of the support area 313 is between the stabilization area 314 and the adjustment area 312. Its wall thickness is at or between 2 to 9 times that of the adjustment area 312, with at least part of the wall thickness being able to be 1.4 mm. However, the thickness of 1.4 mm is still not the minimum required to support the face. Therefore, the support area 313 is configured to have at least two different wall thicknesses, with a thicker part near (adjacent to) the connecting portion 311 to achieve a supportive function. It is specifically divided into the part that is nearer the face (away from the connecting portion 311) and the part that is away from the face (near the connecting portion 311). The part nearer the face, which is thinner, either completely or partially contacts the patient's face during use, while the part away from the face may partially contact the patient's face during use, has a gradually varying wall thickness, and is thicker (relative to the part nearer the face). The boundary at which the two parts connect can be continuous (smoothly connected) or non-continuous (stepped). The top of the support area 313, near the adjustment area 312, has a thicker wall thickness than that of the adjustment area 312 to prevent the patient's nose from bottoming out (being pressed too hard against the rigid section 2) during use. Specifically, the top of the support area 313 near the adjustment area 312 has a gradually varying wall thickness, with the part nearer the connecting portion 311 being thicker, configured to prevent the patient's nose from bottoming out during use. As illustrated in FIG. 13, when the patient's nose presses against the support area 313, the varying wall thickness and the gradual thickening near the connecting portion 311 provide support to the nose, ensuring that the nose does not come into contact with and press against the rigid section 2. The wall thickness at the top of the support area 313 near the adjustment area 312 can also be uniform.

The stabilization area 314, near and connected to the support area 313, has a wall thickness that is thicker than at least part of the wall thickness of both the adjustment area 312 and the support area 313. It is configured to be positioned at the upper end of the patient interface cushion 1 near the nasal side adjustment part 3122, to prevent the elastic section 3 from flipping over under the influence of pressurized gas during use. The stabilization area 314 is the least proportioned area in the main body 31 of the elastic section of the patient interface cushion 1 (i.e., the stabilization area 314 has the smallest area compared to the adjustment area 312 and support area 313), with its wall thickness being at or between 3 to 10 times that of the adjustment area 312. The shape of the stabilization area 314 can be elongated, triangular, or other shapes.

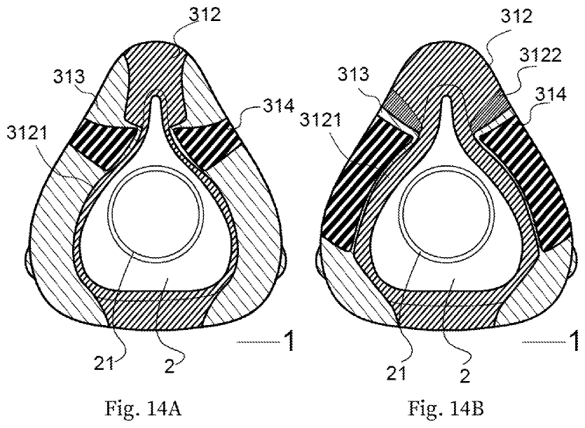

In another embodiment, the patient interface cushion 1, in addition to the regional divisions shown in this disclosure, can have other different regional divisions (as shown in FIGS. 14A-B, where FIG. 14A has no nasal side adjustment part 312, and FIG. 14B has different area sizes for each region).

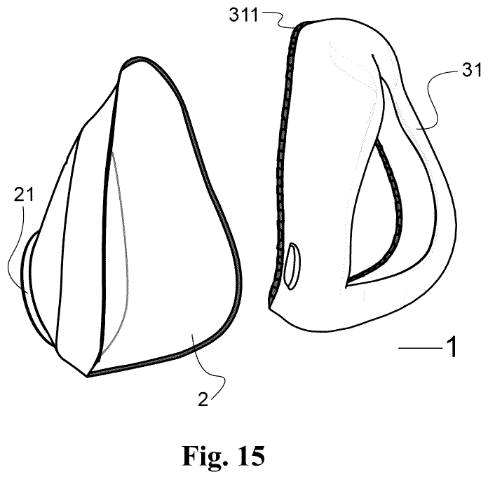

In another embodiment, the elastic section 3 of the patient interface cushion 1 is detachably connectable to the rigid section 2, where the connection method between the elastic section 3 and the rigid section 2 can be a physical clip (as shown in FIG. 15), adhesive connection, magnetic attraction, or other connection methods.

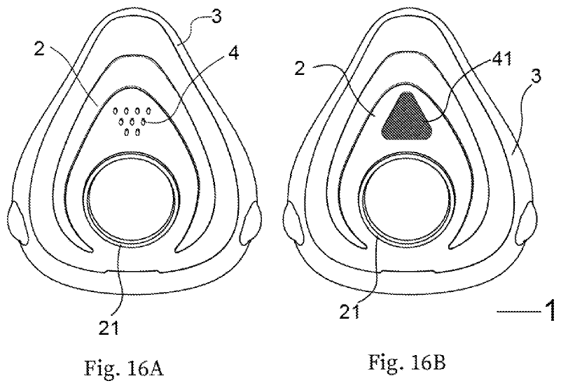

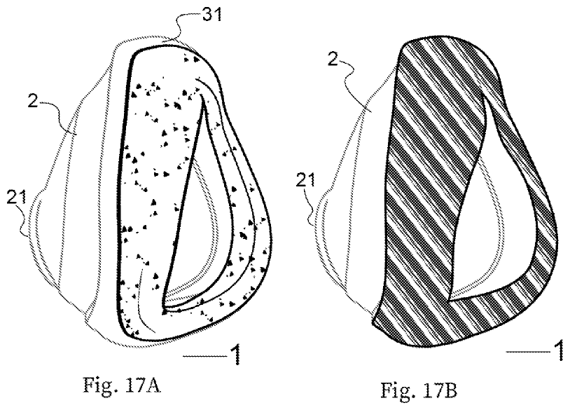

In another embodiment, the patient interface cushion 1 has an exhaust port 4 set on the rigid section 2. And the patient interface cushion 1 can include a noise reduction component 41 (as shown in FIGS. 16A-B, where FIG. 16A shows the patient interface cushion 1 with an exhaust port 4 and FIG. 17B shows the patient interface cushion 1 with a noise reduction component 41). The noise reduction component 41 can be made of noise reduction cotton, noise reduction mesh, or other noise-reducing materials.

In another embodiment, the patient interface cushion 1 may have a comfort layer set on the elastic section 3 to enhance patient comfort. The material of the comfort layer can be foam, textile, or a composite of both (as shown in FIGS. 17A-B which only displays one type of the comfort layer material set on the elastic section 3, where FIG. 17A shows foam material set on the elastic section 3 of the patient interface cushion 1, and FIG. 17B shows textile material). The comfort layer is connectable to the patient interface cushion 1 via a film, adhesive, or physical connection.

### Embodiment 2

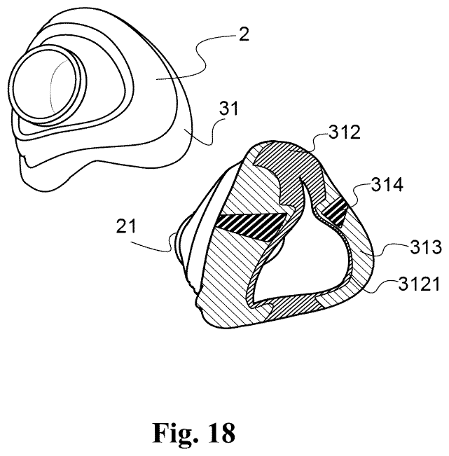

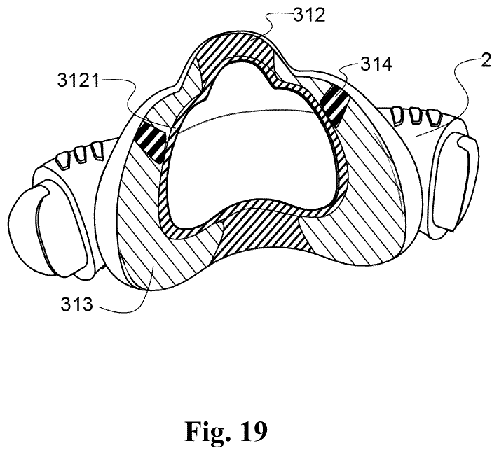

This embodiment provides a patient interface cushion 1 for use in the CPAP field, as shown in FIGS. 18 and 19. It offers a three-dimensional schematic diagram of the patient interface cushion 1. In the embodiment shown in FIGS. 18 and 19, the difference from Embodiment 1 of the patient interface cushion 1 lies in the elastic section 3 being set on another form of patient interface cushion 1. The different areas of the patient interface cushion 1 are the result of experimental testing and comprehensive consideration. The elastic section 3 of the patient interface cushion 1 is not only comfortable to wear but also provides more stable sealing. It can be used for full-face masks and other patient interface cushions, such as nasal masks and oronasal masks, to improve discomfort and poor sealing of other patient interface cushions, ensuring consistency in materials and comfort. Different forms of the patient interface cushion 1 use the same type of regional division and surface treatments, and can achieve similar functions using the same materials, reducing the trial and error costs, storage costs, and management costs of materials. Since the regional division is largely the same, different forms of the patient interface cushion 1 can be obtained simply by changing the composition ratios of different areas, improving the efficiency of research and development and production. Compared to double-layered patient interface cushions and ones with straps, they are also more cost-effective to produce.

### Embodiment 3

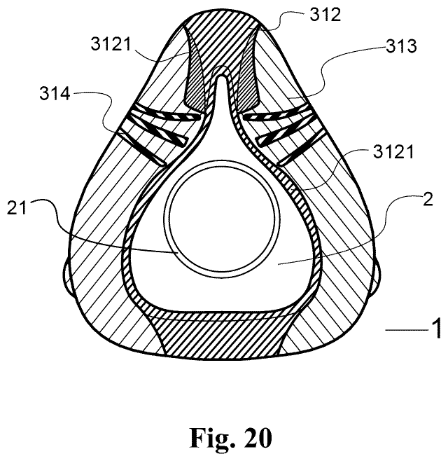

This embodiment provides a patient interface cushion 1 for use in the CPAP field, as shown in FIG. 20. This embodiment offers a three-dimensional schematic diagram of the patient interface cushion 1. In the embodiment shown in FIG. 20, the difference from Embodiment 1 is that the stabilization area 314 of the patient interface cushion 1 has a different form. It is non-continuous, composed of several small areas. Advantages of the patient interface cushions 1 using the same method (dividing into at least three different areas) include: ensuring product consistency in quality and performance while offering different or more diversified patient interface cushions to meet various patient needs. Since the structure of the patient interface cushions is similar, patients can anticipate the performance of similarly divided cushions, increasing their trust in the product. This method increases the diversity of patient interface cushions 1 without complicating production, making the product more competitive on the market and potentially beneficial for both producers and consumers.

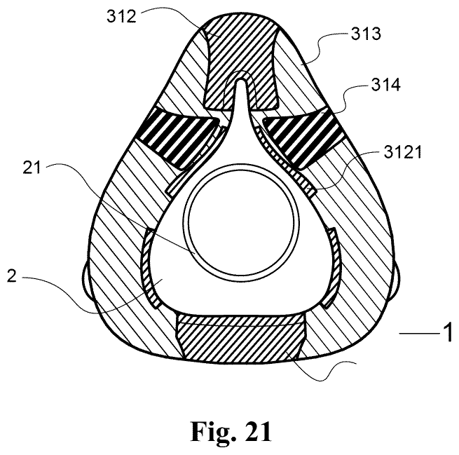

In another embodiment, the adjustment area 312 of the patient interface cushion 1 has different forms (as shown in FIG. 21), with the adjustment area 312 being non-continuous, situated in areas of the face with significant contours or where sensitivity to pressure is heightened.

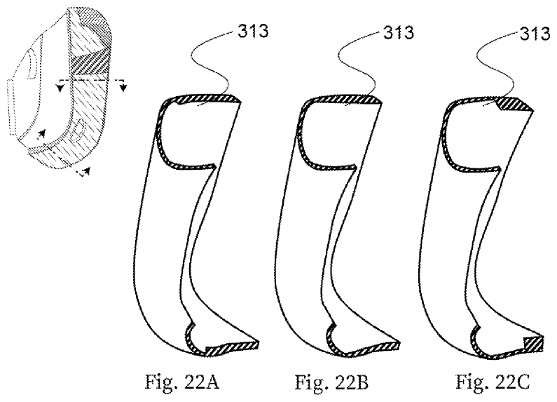

In another embodiment, the support area 313 of the patient interface cushion 1 has different forms. The thicker part (away from the face) and the thinner part (near the face) of the support area 313 have the same area or thickness as those of Embodiment 1, but the variation in wall thicknesses between the thicker part and the thinner part is specified. For example, the support area 313 can consist of a uniformly thinner part near the face and a uniformly thicker part away from the face (as shown in FIG. 22A); or it can consist of a uniformly thinner part near the face and a non-uniform part away from the face, which means that the support area 313 has a gradually varying wall thickness near the connecting portion 311 (as shown in FIG. 22B). Alternatively, as shown in FIG. 22C, the area or thickness of the thicker part and the thinner part of the support area 313 differ from Embodiment 1.

The technical features of the embodiments described above can be combined in any manner. For brevity, not all possible combinations of the technical features in the aforementioned embodiments have been described. However, as long as there is no contradiction in the combination of these technical features, they should be considered within the scope documented in this specification.

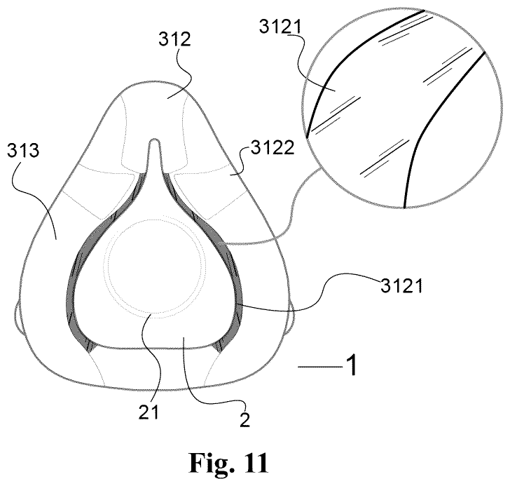

The benefits of a patient interface cushion provided by this disclosure can at least include:
1) By testing the pressure values and sensitivity of different facial areas, combined with the seal of the patient interface cushion, different areas are defined in the main body of the elastic section, each having specific functions. This provides higher comfort and a better user experience for the patient, thus improving their compliance with the treatment. Through research on platforms like CPAP forums, Reddit, Amazon customer review sections, YouTube, etc., it was found that most existing patient interface cushions on the market cause discomfort or leakage during wear. During wear, discomfort in patients is caused not only by the material of the patient interface cushion but also by its inability to fully conform to the patient's face. As different areas of the face have distinct contour curves, existing patient interface cushions on the market cannot adjust and vary according to these facial contour curves. Consequently, they fail to provide satisfactory comfort and pressure for the patient. This inadequacy in fitting also leads to insufficient sealing of the patient interface cushions. When the same amount of pressure is applied to the elastic section of the patient interface cushion, the elastic section deforms uniformly to fit the face. However, due to the varying contour curves of the face, this uniform deformation of the elastic section cannot achieve a complete fit with the patient's face, resulting in air leakage. And existing patient interface cushions on the market have parts that are easily blown over, so the parts were thickened to prevent turning out and leakage. In summary, based on precise experimental data, this disclosure divides the main body of the elastic section of the patient interface cushion, which has the most contact with the patient's face, into at least three areas: an adjustment area, a stabilization area, and a support area. The adjustment area is configured to increase compatibility with the patient's face and nose, the stabilization area is thickened to prevent the elastic section from turning out, and the support area is to support the patient interface cushion. To ensure the supportive function of the elastic section of the patient interface cushion, the support area has at least two different thicknesses, with the part near the connecting portion being thicker and variable in wall thicknesses, and the part nearer the patient's face being thinner during use.
2) In the adjustment area, a thinner edge portion is set to increase compatibility with the patient's face, making the elastic section of the patient interface cushion fit more closely to the patient's face and enhancing the sealing. Based on the above experiments, this disclosure noted that the main body of the elastic section of the patient interface cushions available on the market greatly impacts the overall sealing and comfort of the cushion, particularly in the edge portion near the opening facing the patient's face. Therefore, the edge portion has been reconfigured in the disclosure to improve the comfort and sealing while making the patient interface cushion more suitable for most patients. a) From some Amazon buyer reviews and comments on other platforms, this disclosure found that although existing patient interface cushions on the market come in S, M, L sizes, due to differences in race and weight, some patients still cannot find a cushion that fits their face size, with the L size causing gas leakage and the M size creating discomfort due to pressure. Hence, the disclosure has made the edge portion near the opening facing the patient's face of the main body of the elastic section of the patient interface cushion thinner to accommodate more face shapes. With the thinner edge portion that can easily fold inward, patients with smaller faces can directly use the inner edge as the sealing edge under the pressurized gas, while patients with larger faces can press down the edge portion of the patient interface cushion and use the edge of the support area near the edge for sealing. This disclosure adapts the patient interface cushion to a range of face sizes instead of a specific value, facilitating the selection for patients who cannot find a suitable size among various brands, thereby expanding the compatibility of the patient interface cushion. b) In addition to increasing compatibility, setting the edge portion to be thinner also enhances the sealing of the patient interface cushion. The design which makes the edge portion thin and of a uniform thickness allows the edge portion to be more easily lifted by airflow during use, facilitating contact with the patient's face to form a seal, making the seal more stable. The thinner edge portion has greater flexibility and can better adapt to different facial contours. Uniform thickness means that the force exerted on the patient's face by the inner edge of the patient interface cushion is more even, preventing excessive pressure points or discomfort, thereby providing more uniform comfort. Also, the uniform thickness can accommodate more facial contour shapes, fitting a wider range of people. c) This disclosure uses a special surface treatment process, such as, a polishing of the edge portion, as shown in FIG. 11. A polished surface is smoother than other surfaces and has greater adhesion to the skin during use, thus achieving a better and more stable fit. Moreover, a polished surface is more uniform, without depressions or protrusions, increasing the sealing between the elastic section of the patient interface cushion and the patient's face and reducing gas leakage. To ensure comfort, the patient interface cushion also has a relatively rough frosted surface, which has less adhesion to the skin during use, increasing comfort for the patient wearing the patient interface cushion. This disclosure combines two surface treatments to ensure better sealing while maintaining the comfort of the patient interface cushion.
3) The design of the area near the nasal airway is easily adjustable to increase the accommodation of the patient's nose and prevent bottoming out. One of the significant differences in human faces lies in the height and width of the nose. Some people have higher nose bridges, while others have lower ones; some have narrower noses, and others have wider noses. Such differences lead to the necessity of adapting the patient interface cushion to a variety of nose heights and widths. The design of the area at which the main body of the elastic section contacts the patient's alae nasi directly affects the fit and sealing effect of the patient interface cushion with the patient's face and it should be carefully considered. The patient interface cushion in this disclosure has at least two different wall thicknesses in the area where the main body of the elastic section contacts the patient's alae nasi and nasal sides. This design allows greater accommodation for the nose during use, adapting to various nose heights and widths, and further increasing the compatibility of the patient interface cushion with the patient's face, thus providing better treatment efficacy. Specifically, a) when patients use the patient interface cushion of this disclosure, the area of the main body of the elastic section that has the most contact with the patient's nose is the adjustment area with a thinner wall thickness. Its thinner wall can more easily conform to the patient's nose. The adjustment area can easily deform according to the height of the nose due to the pressure from the nose, and it has a thinner wall thickness, which brings greater flexibility and less pressure on the nose, improving comfort during use. b) The support area, near the top of the adjustment area, has a thicker wall thickness than that of the adjustment area. It is configured to prevent the elastic section from bottoming out due to excessive pressure from the patient's nose, providing greater support for the nose and reducing discomfort caused by the patient interface cushion. c) The adjustment area near the patient's nose also includes a nasal side adjustment part. To address the issue of patient interface cushions not fitting patients with different nose widths, the nasal side adjustment part enhances the compatibility of the patient interface cushion with the patient's nose. Under the pressurized gas, patients with narrower noses can use the boundary of the thinner area as the outer contour of the elastic section of the patient interface cushion to accommodate the nose and change the shape. In contrast, patients with wider noses can press down the nasal side adjustment part, using part of or the entire boundary of the nasal side adjustment part as the outer contour of the elastic section of the patient interface cushion to accommodate the nose and change the shape. This design adapts to patients with different nose widths, ensures a better fit of the patient interface cushion with the patient's face and meets the needs of various patients.
4) Compared to patient interface cushions with straps and dual-layer designs, this disclosure maintains the original functionality while simplifying the mold, reducing the defect rate, and lowering production costs. It also offers benefits in terms of environmental protection and ease of cleaning. Most existing patient interface cushions on the market have a double-layer structure or straps to prevent the elastic section from turning out. While these designs solve the issue of the elastic section turning out, they introduce several problems. For example, double-layered patient interface cushions are heavier than single-layered ones, which can cause discomfort for patients, especially during long periods of wear. Both double-layered patient interface cushions and ones with straps are more expensive in terms of material costs, require more complex molds leading to higher mold costs, and involve more complicated manufacturing processes. Additionally, due to the complexity of their processes, the likelihood of producing defective products increases during manufacturing, which further elevates manufacturing costs and may result in increased labor costs due to quality control and sorting. The patient interface cushion provided by this disclosure uses the stabilization area of the main body of the elastic section to replace the structure of double-layered patient interface cushions or ones with straps, achieving the same functionality but with simpler, more cost-effective processes and a higher yield of quality products. This design demonstrates better features compared to existing patient interface cushions in various aspects and meets the requirements of green design, having beneficial effects on climate change and environmental protection. In terms of cleaning, double-layered patient interface cushions or ones with straps, being made of multiple layers of material, may have corners that easily accumulate dirt and are hard to clean. Over time, this can lead to the growth of bacteria and other microbes, adversely affecting patient health. The cleaning process may require more time and possibly more frequent cleaning sessions to ensure thoroughness. Moreover, during the use of ventilators for the treatment of OSA, the patient interface cushions tend to accumulate moisture. The multi-layer material impedes evaporation of the moisture and requires longer drying times, which is not conducive to multiple or prolonged usage by patients and is another factor detrimental to patients' health.

The embodiments of the disclosure are described above in conjunction with the accompanying drawings, but the disclosure is not limited to the embodiments described above. The specific embodiments are merely illustrative and not restrictive. Those of ordinary skills in this field, in light of the teachings of the disclosure and without departing from the spirit and scope of the disclosure as defined by the claims, can develop numerous forms that fall within the protection of the disclosure.

It must be noted that as used herein and in the appended claims, the singular forms “a”, “an”, and “the” include their plural equivalents, unless the context clearly dictates otherwise.

1. A patient interface cushion, configured to deliver breathable pressurized gas to a patient to treat sleep breathing disorders, the patient interface cushion comprising:
a rigid section having an air intake opening to receive breathable pressurized gas, the rigid section being configured to connect to an elastic section at one end;
the elastic section, molded together with the rigid section, including:
a main body that is configured to at least partially contact and form a seal with a face of the patient during use, and
a connecting portion that is configured to connect to the rigid section;
wherein the main body of the elastic section has at least three different wall thicknesses, the main body of the elastic section comprising at least:
an adjustment area, with an opening near the face of the patient and an edge portion near the opening, wherein the edge portion near the opening is configured to:
fold inward to seal at the patient's face, and
when a patient's face is smaller, receive a pressurized gas to expand, or when a patient's face is larger, be pressed down;
a support area, configured to connect to the connecting portion of the elastic section, at least two different wall thicknesses, and a part near the connecting portion has a thicker wall thickness for support; and
a stabilization area, near and connected to the support area, by having a wall thickness thicker than at least part of a wall thickness of the support area.

2. (canceled)

3. The patient interface cushion according to claim 1, wherein the adjustment area, during use, surrounds a nasal airway and an oral airway of the patient.

4. The patient interface cushion according to claim 1, wherein the stabilization area is a least proportioned area in the main body of the elastic section of the patient interface cushion.

5. The patient interface cushion according to claim 1, wherein each area of the main body of the elastic section is smoothly connected, without any obvious demarcation lines.

6. A patient interface cushion, configured to deliver breathable pressurized gas to a patient to treat sleep breathing disorders, the patient interface cushion comprising:
a rigid section having an air intake opening to receive breathable pressurized gas, the rigid section being configured to connect to an elastic section at one end;
the elastic section with a single-layer wall, molded together with the rigid section, including a main body that is configured to at least partially contact and form a seal with a face of the patient during use, and a connecting portion to connect to the rigid section;
wherein the main body of the elastic section comprises:
an adjustment area, with an opening near the face of the patient and an edge portion near the opening, wherein the edge portion near the opening is configured to:
fold inward to seal at the patient's face, and
when a patient's face is smaller, receive a pressurized gas to expand, or when a patient's face is larger, be pressed down;
a support area, configured to connect to the connecting portion of the elastic section, the support area having at least two different wall thicknesses including a wall thickness between 2 to 9 times a wall thickness of the adjustment area and a part near the connecting portion has a thicker wall thickness for support; and
a stabilization area, near and connected to the support area, with a wall thickness between 3 to 10 times the wall thickness of the adjustment area.

7. The patient interface cushion according to claim 6, wherein the wall thickness of the adjustment area is at least 0.3 mm.

8. The patient interface cushion according to claim 6, wherein a width of the edge portion of the adjustment area is at most 2 cm.

9. The patient interface cushion according to claim 6, wherein each area of the main body of the elastic section has a uniform wall thickness.

10. The patient interface cushion according to claim 6, wherein the main body of the elastic section has abrupt changes in wall thicknesses between its areas, with distinct demarcation lines between each area.

11. A patient interface cushion, configured to deliver breathable pressurized gas to a patient to treat sleep breathing disorders, the patient interface cushion comprising:
a rigid section having an air intake opening to receive breathable pressurized gas, the rigid section being configured to connect to an elastic section at one end;
the elastic section, molded together with the rigid section, including a main body that is configured to at least partially contact and form a seal with a face of the patient during use, and a connecting portion to connect to the rigid section;
the main body of the elastic section having at least three different wall thicknesses and at least two surface treatments, the main body of the elastic section at least comprising:
an adjustment area, with an opening near the face of the patient and an edge portion near the opening, wherein
at least part of the edge portion has a surface treatment of polishing, and
the edge portion near the opening is configured to:
fold inward to seal at the patient's face, and
when a patient's face is smaller, receive a pressurized gas to expand, or when a patient's face is larger, be pressed down;
a support area, configured to connect to the connecting portion of the elastic section, having a gradually varying wall thickness that is thicker than a wall thickness of the adjustment area at a top near the adjustment area to prevent a nose of the patient from bottoming out during use and also has a thicker wall thickness near the connecting portion; and
a stabilization area, near and connected to the support area, with a wall thickness thicker than the wall thickness of the adjustment area.

12. (canceled)

13. The patient interface cushion according to claim 11, wherein a maximum width of the adjustment area at a top of the patient interface cushion to accommodate the nose does not exceed 5.5 cm.

14. The patient interface cushion according to claim 11, wherein part of the adjustment area that contacts the nose of the patient during use has an inward concave, configured to better accommodate the nose and form a seal.

15. The patient interface cushion according to claim 11, wherein the stabilization area is a least proportioned area in the main body of the elastic section of the patient interface cushion.

16. The patient interface cushion according to claim 11, wherein the adjustment area includes a nasal side adjustment part with a wall thickness between a wall thickness of the edge portion and the support area.

17. A patient interface cushion, configured to deliver breathable pressurized gas to a patient to treat sleep breathing disorders, the patient interface cushion comprising:
a rigid section having an air intake opening to receive breathable pressurized gas, the rigid section being configured to connect to an elastic section at one end;
the elastic section with a single-layer wall, molded together with the rigid section, including a main body that is configured to at least partially contact and form a seal with a face of the patient during use, and a connecting portion that is configured to connect to the rigid section;
wherein the main body of the elastic section comprises:
an adjustment area, with an opening near the face of the patient and an edge portion near the opening, and
a nasal side adjustment part near the edge portion with a wall thickness between a wall thickness of the edge portion and a support area,
wherein the adjustment area has at least part of a wall thickness of at least 0.3 mm, and
wherein the edge portion near the opening is configured to:
fold inward to seal at the patient's face, and
when a patient's face is smaller, receive a pressurized gas to expand, or when a patient's face is larger, be pressed down;
the support area, configured to connect to the connecting portion of the elastic section, having at least two different wall thicknesses, and a part near the connecting portion having a gradually varying wall thickness for support; and
a stabilization area, near and connected to the support area.

18. The patient interface cushion according to claim 17, wherein the stabilization area has a wall thickness thicker than at least part of a wall thickness of the adjustment area and the support area.

19. (canceled)

20. The patient interface cushion according to claim 17, wherein the elastic section has at least three different wall thicknesses.

21. The patient interface cushion according to claim 17, wherein each area of the main body of the elastic section is smoothly connected, without any obvious demarcation lines.

22. The patient interface cushion according to claim 17, wherein a width of the edge portion of the adjustment area is at most 2 cm.
